(19) United States
(12) Patent Application Publication
(10) Pub. No.: US 2020/0352693 A1
(43) Pub. Date: Nov. 12, 2020

(54) PACKAGE FOR THE CONTAINMENT, HANDLING, AND DELIVERY OF INTERBODY CAGES

(71) Applicant: SPINAL BALANCE, INC., Swanton, OH (US)

(72) Inventors: Faezah Agarwal, Swanton, OH (US); Aakash Agarwal, Swanton, OH (US)

(73) Assignee: SPINAL BALANCE, INC., Swanton, OH (US)

(21) Appl. No.: 16/765,898

(22) PCT Filed: Dec. 7, 2018
(86) PCT No.: PCT/US18/64421

§ 371 Date: May 21, 2020

Related U.S. Application Data
(60) Provisional application No. 62/597,098, filed on Dec. 11, 2017.

(51) Int. Cl. A61F 2/00; A61F 2/44; A61F 2/46

(52) U.S. Cl. CPC A61F 2/0095; A61F 2/4611; A61F 2/442

(57) ABSTRACT

A method of delivering a medical device to a desired location at a surgical site includes the initial steps of providing a package including a hollow body that extends from a first opened end to a second opened end, and providing a medical device within the hollow body between the first opened end and the second opened end. The package is oriented such that the first opened end is disposed adjacent to a desired location at a surgical site. Lastly, the medical device is moved through the first opened end from the hollow body to the desired location.

### CROSS-REFERENCE TO RELATED APPLICATIONS

This application claims the benefit of U.S. Provisional Application No. 62/597,098, filed Dec. 11, 2017, the disclosure of which is incorporated herein by reference.

### BACKGROUND OF THE INVENTION

This invention relates in general to packaging for medical devices. In particular, this invention relates to an improved structure for a package for containing, handling, and delivering an interbody cage or other medical device to a desired site, such as between two adjacent vertebrae of a spinal column during a surgical procedure.

Interbody cages are medical implants that are designed to be inserted between two bones during a surgical procedure. For example, an interbody cage may be disposed between adjacent vertebrae of a spinal column for height restoration and mechanical support to facilitate fusion between the two bones. In some instances, a plurality of interbody cages of varying sizes are provided to the surgeon in a preprocessed tray. In other instances, individual interbody cages are provided to the surgeon in respective pre-sterilized packages, which are opened for use. Regardless of the manner in which an interbody cage is initially provided to the surgeon, it must subsequently be grasped by the fingers of the surgeon in order to manually pack it with a bone graft material and attach it to a delivery instrument. The interbody cage then is positioned between the adjacent vertebrae of the spinal column, usually through vigorous impaction.

There are four disadvantages to this manually intensive process, namely, reduced asepsis, elaborate delivery time, the potential for nerve root and spinal cord damage using a dedicated retractor to limit the amount of touching between the cage and the nerves, and the dislodgement of cage followed by other cascade of events such as delayed surgery and nerve root damage. The problem of asepsis arises from intraoperative handling of the interbody cage (and other devices) that are to be implanted into the body. The gloves worn by the surgeon are known to harbor bacteria (such as from wound site), and it has been proven that reducing the number of touches or other manual handling reduces the rate of infection after such surgeries. Another major cause of asepsis is direct contact with the wound site during delivery. Other disadvantages are related to the delivery mechanism and may lead to surgically-related neurological deficit complications and can result in a direct increase in cost, as every minute of surgical time adds to the total cost that the patient and the healthcare system incur from the given procedure.

### SUMMARY OF THE INVENTION

This invention relates to an improved structure for a package for containing, handling, and delivering an interbody cage or other device to a desired location, such as between two adjacent vertebrae of a spinal column during a surgical procedure. The invention provides a utility to remove the above disadvantages from the system. For users, it provides saving in surgical time, better ergonomics, and ease and assistance during surgeries. For patients, it allows for less blood loss, superior asepsis, less chance of post-surgery infection, overall safer procedures with no neurological deficit (as the delivery is through a tube-like feature that has no sharp features and does not retract the spinal cord and nerve tissue more than necessary), and expedited healing.

The invention is a protective package that allows for the no-touch handling and delivery of the interbody cages to the surgical site. In other words, the implant itself is not directly touched by the user during the installation process. The invention allows the user (i.e., a surgeon or an assistant) to effortlessly, handle, and deliver the interbody cage to the surgical site without contaminating it through touching with either gloves or fingers, and further without the interbody cage coming in direct contact with the wound site. The invention also helps to reduce impaction and forced delivery of the interbody cages by means of features that streamline the path of entry. In addition, because of the inherent nature of this type of delivery, the nerve tissue and spinal cord can be protected from damage, as only the smooth surface (in some embodiments) comes in contact therewith and not the actual device.

In its most basic embodiment, the package is a hollow elongated structure with openings on both ends (i.e., proximal and distal ends) and with a space inside to contain and deliver the device between two bony structures. The distal end has contours and a surface profile that allow for assistive movement desirable for delivery, whereas the proximal end has an access point for pushing the cage forward. The proximal end also has features that allow for the stable attachment of another instrument if needed for ergonomic or technique specific purpose.

Various aspects of this invention will become apparent to those skilled in the art from the following detailed description of the preferred embodiments, when read in light of the accompanying drawings.

### BRIEF DESCRIPTION OF THE DRAWINGS

FIG. 1 is a perspective view of a first embodiment of a package in accordance with this invention for containing, handling, and delivering an interbody cage between adjacent vertebrae of a spinal column during a surgical procedure.

FIG. 2 is a sectional perspective view of the first embodiment of the package illustrated in FIG. 1.

FIG. 3 is a sectional perspective view similar to FIG. 2 showing an interbody cage disposed within the first embodiment of the package.

FIG. 4 is a sectional elevational view showing the first embodiment of the package and the interbody cage initially aligned between the adjacent vertebrae of the spinal column.

FIG. 5 is a sectional elevational similar to FIG. 4 showing the first embodiment of the package and the interbody cage disposed next to the adjacent vertebrae of the spinal column, together with a tool for inserting the interbody cage between the adjacent vertebrae.

FIG. 6 is a sectional elevational view similar to FIG. 5 showing the interbody cage partially inserted between the adjacent vertebrae of the spinal column.

FIG. 7 is a sectional elevational view similar to FIG. 6 showing the interbody cage fully inserted between the adjacent vertebrae of the spinal column.

FIG. 8 is a perspective view of a second embodiment of a package in accordance with this invention for containing, handling, and delivering an interbody cage between adjacent vertebrae of a spinal column during a surgical procedure.

FIG. 9 is a perspective view of a third embodiment of a package in accordance with this invention for containing, handling, and delivering an interbody cage between adjacent vertebrae of a spinal column during a surgical procedure.

### DETAILED DESCRIPTION OF THE PREFERRED EMBODIMENTS

Referring now to the drawings, there is illustrated in FIGS. 1 through 7 a first embodiment of a package, indicated generally at 10, in accordance with this invention. The first embodiment of the package 10 will be described and illustrated in the context of containing, handling, and delivering an interbody cage 15 (see FIGS. 3 through 7) between adjacent vertebrae V1 and V2 of a spinal column during a surgical procedure. However, this invention may be used in any desired environment for any desired purpose.

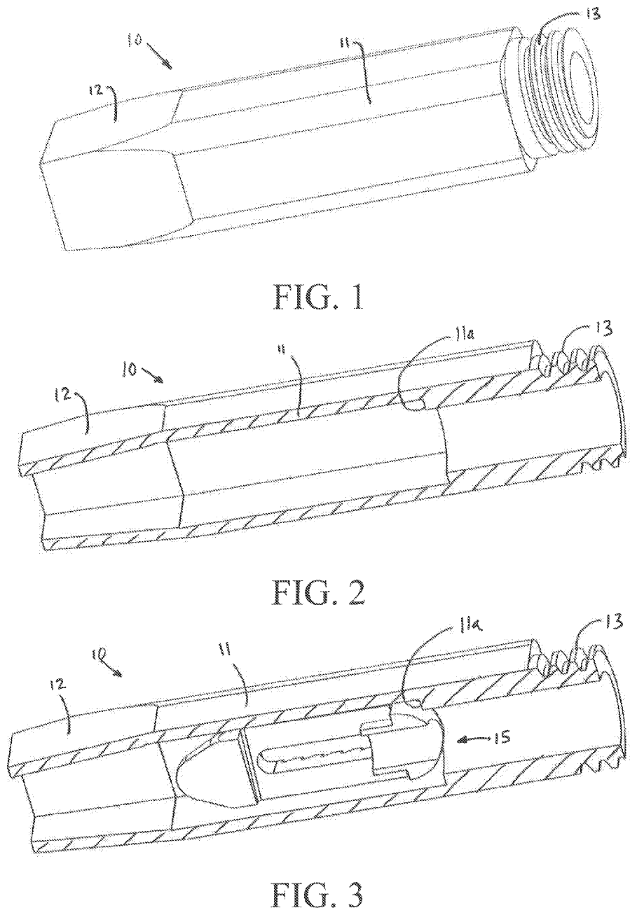

As best shown in FIGS. 1 and 2, the first embodiment of the package 10 includes an elongated hollow body 11 that extends from a first opened end 12 to a second opened end 13. In the illustrated embodiment, the hollow body 11 is formed by a wall that is generally hollow and cylindrical in shape, having a plurality of flats provided about the circumference thereof to facilitate the handling thereof by a user, such as a surgeon or an assistant. However, the hollow body 11 of the first embodiment of the package 10 may be formed having any desired shape. The first embodiment of the package 10 is preferably formed from a relatively flexible material, such as a plastic material. However, the first embodiment of the package 10 may be formed from any desired material or combination of materials. In the illustrated embodiment, the hollow body 11 extends generally linearly from the first opened end 12 to the second opened end 13. However, the hollow body 11 may be formed having one or more corrugated portions (not shown) or other similar structures to provide additional flexibility to the hollow body 11, for a purpose that will be explained below.

An interior surface of the hollow body 11 may optionally be provided with an internal shoulder 11a adjacent to the second opened end 13. The illustrated first end 12 of the first embodiment of the package 10 tapers from a relatively larger dimension defined by the hollow body 11 to a relatively smaller dimension at a tip thereof, although such is not required. The illustrated second end 13 of the first embodiment of the package 10 is provided with an attachment feature, such as the illustrated externally threaded portion, although again such is not required. The purpose of the internal shoulder 11a, the tapered first end 12, and the externally threaded second end 13 of the first embodiment of the package 10 will also be explained below.

As mentioned above, the illustrated first embodiment of the package 10 is generally hollow and cylindrical in shape, defining an interior space within which the interbody cage 15 may be disposed, as shown in FIG. 3. The illustrated interbody cage 15 is intended to be representative of any medical or other device that can be contained, handled, and delivered in the general manner described herein. The interbody cage 15 may be positively retained within this interior space by engagement with the internal shoulder 11 a defined within the hollow body 11 and a reduced inner diameter portion defined within the tapered first end 12. Although not required, the first embodiment of the package 10 and the interbody cage 15 is preferably pre-assembled in the configuration shown in FIG. 3, sterilized as a unit, and disposed in a protective container (not shown) for delivery to the site of the operation for use. Such pre-assembly and sterilization facilitate the insertion of the interbody cage 15 between the adjacent vertebrae V1 and V2 in the manner described below, without any manual handling of the interbody cage 15 by the surgeon or assistant. As is well known, it is very important to minimize or eliminate such manual handling of the interbody cage 15 (or any other medical implant, for that matter) to reduce the likelihood of undesirable contamination.

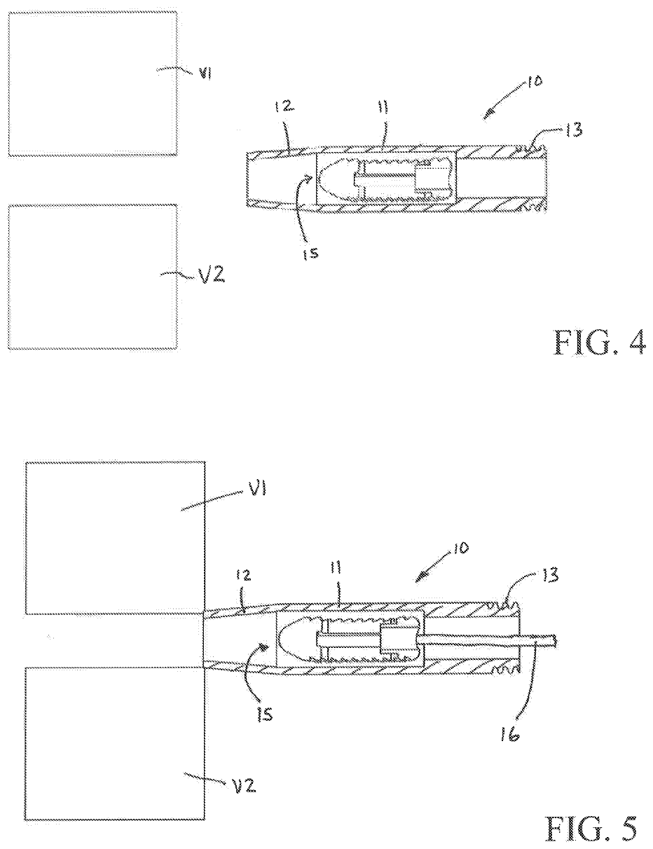

FIGS. 4 through 7 illustrate the steps in a method of delivering the interbody cage 15 from the first embodiment of the package 10 to a space that is defined between the adjacent vertebrae V1 and V2 of the spinal column during a surgical procedure. Initially, the combined assembly of the first embodiment of the package 10 and the interbody cage 15 is removed from the protective container at the site of the operation. Then, as shown in FIG. 4, the combined assembly of the first embodiment of the package 10 and the interbody cage 15 is oriented such that the first end 12 thereof is aligned with the space defined between the adjacent vertebrae V1 and V2. Such alignment can be facilitated by attaching a tool (not shown) to the attachment feature provided on the second end 13 of the first embodiment of the package 10. As mentioned above, this attachment feature may be embodied as an externally threaded portion provided on the second end 13. However, the attachment feature may be embodied as any other desired structure, such as a snap fit or other type of mechanical adapter.

Regardless, as shown in FIG. 5, the combined assembly of the first embodiment of the package and the interbody cage 15 is then disposed adjacent to the space defined between the adjacent vertebrae V1 and V2 such that the first end 12 is aligned with such space. As mentioned above, the first end 12 of the first embodiment of the package 10 preferably tapers from a relatively large dimension defined by the hollow body 11 to a relatively small dimension defined at the tip thereof. The tapered first end 12 not only facilitates this alignment, but also reduces tissue retraction and provides a good gripping relationship between the adjacent vertebrae V1 and V2.

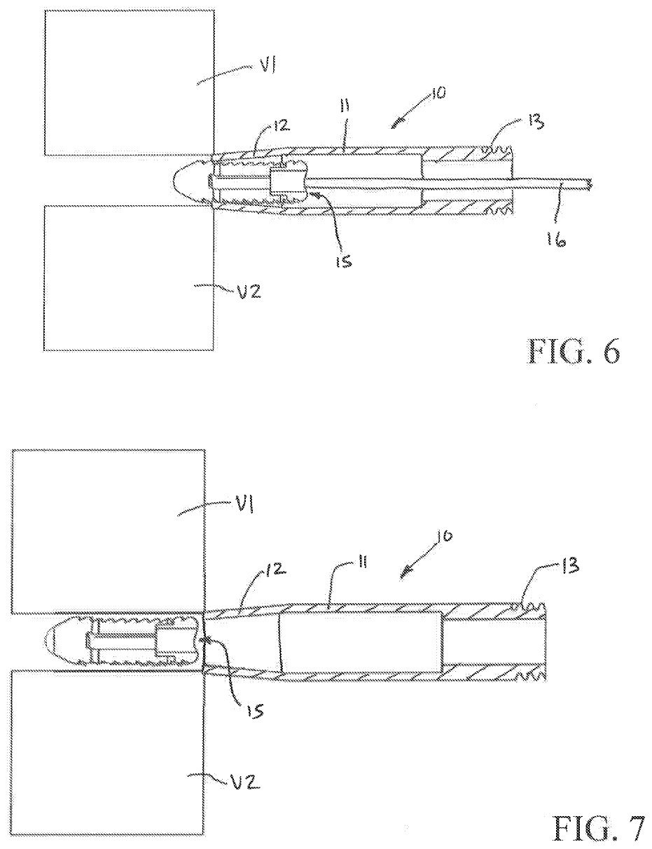

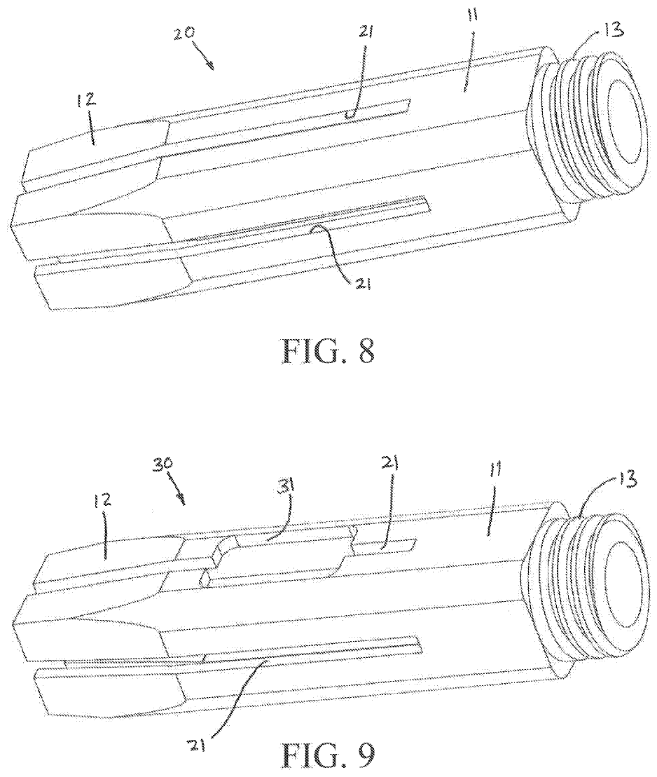

Next, as shown in FIG. 5, a tool 16 is inserted axially through the second end 13 of the first embodiment of the package 10. The tool 16 is moved axially into engagement with the interbody cage 15. Such axial movement of the tool 16 is then continued such that interbody cage 15 is moved axially through the interior space defined by the hollow body 11 of the first embodiment of the package 10. As a result, the interbody cage 15 is moved axially toward and through the opened first end 12 of the first embodiment of the package 10, as shown in FIG. 6. When the interbody cage 15 has been fully ejected from the interior space of the first embodiment of the package 10, it is disposed in the space between the adjacent vertebrae V1 and V2, as shown in FIG. 7. Lastly, the tool 16 is removed from the first embodiment of the package 10, and the first embodiment of the package 10 is removed from the adjacent vertebrae V1 and V2.

As mentioned above, the first embodiment of the package 10 may be formed from a flexible material, and the hollow body 11 may be formed having one or more corrugated portions (not shown) or other similar structures to provide additional flexibility to the hollow body 11. Such flexibility may facilitate the axial movement of the interbody cage 15 by allowing the surgeon or other user to deform the opposed sides of the hollow body 11 inwardly toward one another, thus mechanically engaging a trailing end of the interbody cage 15 (the right end when viewing FIGS. 3 through 7) and urging it for such axial movement. Regardless, however, it can be seen that throughout this entire process, no manual handling of the interbody cage 15 by the surgeon or assistant occurs. As discussed above, the lack of such manual handling is very important to reduce the likelihood of undesirable contamination of the interbody cage 15.

FIG. 8 is a perspective view of a second embodiment of a package, indicated generally at 20, in accordance with this invention. The second embodiment of the package 20 is, in large measure, identical to the first embodiment of the package 10, and like reference numbers are used to indicate similar structures. However, in the second embodiment of the package 20, at least one slot 21 is provided in either or both of the hollow body 11 and the first end 12 thereof. In the illustrated embodiment, four of such slots 21 (only two of which are illustrated) are provided, all of which extend throughout both the hollow body 11 and the opened first end 12 of the second embodiment of the package 20. However, a greater or lesser number of such slots 21 may be provided as desired, and all of such slots 21 need not extend throughout both the hollow body 11 and the first end 12 of the second embodiment of the package 20. The purpose of such slots 21 is to provide additional flexibility to facilitate the movement through the hollow body 11 and ejection of the interbody cage 15 from the first end 12, as described above.

FIG. 9 is a perspective view of a third embodiment of a package, indicated generally at 30, in accordance with this invention. The third embodiment of the package 30 is, in large measure, identical to the second embodiment of the package 20, and like reference numbers are used to indicate similar structures. However, in the third embodiment of the package 30, an enlarged opening 31 is additionally provided in the hollow body 11. In the illustrated embodiment, only one such enlarged opening 31 is provided. However, a greater number of such openings 31 may be provided as desired. The purpose of such openings 31 is to facilitate the insertion of a material, such as a conventional bone graft material, into the interbody cage 15 for the purpose of promoting fusion of the adjacent vertebrae V1 and V2 after the interbody cage 15 has been disposed therebetween.

The principle and mode of operation of this invention have been explained and illustrated in its preferred embodiments. However, it must be understood that this invention may be practiced otherwise than as specifically explained and illustrated without departing from its spirit or scope.

What is claimed is:

1. A method of delivering a medical device to a desired location at a surgical site comprising the steps of:
(a) providing a package including a hollow body that extends from a first opened end to a second opened end;
(b) providing a medical device within the hollow body between the first opened end and the second opened end;
(c) orienting the package such that the first opened end is disposed adjacent to a desired location at a surgical site; and
(d) moving the medical device through the first opened end from the hollow body to the desired location.

2. The method defined in claim 1 wherein step (a) is performed by providing a package wherein the first opened end tapers from a relatively larger dimension defined by the hollow body to a relatively smaller dimension at a tip thereof.

3. The method defined in claim 1 wherein step (a) is performed by providing a package wherein the second opened end is provided with an attachment feature.

4. The method defined in claim 1 wherein step (a) is performed by providing a package wherein an interior surface of the hollow body is provided with an internal shoulder adjacent to the second opened end.

5. The method defined in claim 1 wherein step (a) is performed by providing a package wherein the first opened end tapers from a relatively larger dimension defined by the hollow body to a relatively smaller dimension at a tip thereof, the second opened end is provided with an attachment feature, and an interior surface of the hollow body is provided with an internal shoulder adjacent to the second opened end.

6. The method defined in claim 1 wherein step (a) is performed by providing a package wherein the hollow body has one or more slots provided therein.

7. The method defined in claim 2 wherein step (a) is performed by providing a package wherein the hollow body and the tapered opened first end has one or more slots provided therein.

8. The method defined in claim 7 wherein step (a) is performed by providing a package wherein the hollow body additionally has an opening provided therein.

9. The method defined in claim 1 wherein step (d) is performed by inserting a tool axially through the second opened end into engagement with the medical device.

10. The method defined in claim 1 wherein step (d) is performed by deforming opposed sides of the hollow body inwardly toward one another and into engagement with the medical device.

11. A combined package and medical device for containing, handling, and delivering the medical device to a surgical site comprising:
a package including a hollow body that extends from a first opened end to a second opened end; and
a medical device disposed within the hollow body between the first opened end and the second opened end.

12. The combined package and medical device defined in claim 11 wherein the first opened end tapers from a relatively larger dimension defined by the hollow body to a relatively smaller dimension at a tip thereof.

13. The combined package and medical device defined in claim 11 wherein the second opened end is provided with an attachment feature.

14. The combined package and medical device defined in claim 11 wherein an interior surface of the hollow body is provided with an internal shoulder adjacent to the second opened end.

15. The combined package and medical device defined in claim 11 wherein the first opened end tapers from a relatively larger dimension defined by the hollow body to a relatively smaller dimension at a tip thereof, the second opened end is provided with an attachment feature, and an interior surface of the hollow body is provided with an internal shoulder adjacent to the second opened end.

16. The combined package and medical device defined in claim 11 wherein the hollow body has one or more slots provided therein.

17. The combined package and medical device defined in claim 12 wherein the hollow body and the tapered opened first end has one or more slots provided therein.
